SEQUENCE LISTING (1 sequence)

SEQ ID NO: 1 - protein, 240 aa, Saccharomyces cerevisiae
MADEERLKEFKEANKIVFDPNTRQVWENQNRDGTKPATTFQSEEDMKRAAPESEKGTSATSGIVPTLQNIVATVTLGCRLDLKTVALHARNAEYNPKRFAAVIMRIREPKTTALIFAPGKMVVTGAKSEDDSKLASRKYARIIQKIGFAAKFTDFKIQNIVGSCDVKFPIRLEGLAFSHGTFSSYEPELFPGLIHRMVKPKIVLSIFVSGKIVLTGAKQREEIYQAFEAIYPVLSEFRKM

(19) United States
(12) Patent Application Publication
(10) Pub. No.: US 2026/0317667 A1
(43) Pub. Date: Oct. 15, 2026

(54) A BEVERAGE

(71) Applicant: JUICY RIVER LTD, London (GB)

(72) Inventors: Roberto Vanin, London (GB); Claudia Borgmeier, Zwingenberg (DE)

(21) Appl. No.: 19/483,730

(22) PCT Filed: May 10, 2024
(86) PCT No.: PCT/GB2024/051218

§ 371 Date: Nov. 12, 2025

(30) Foreign Application Priority Data
May 12, 2023 (GB) 2307117.8

(51) Int. Cl. C12G 1/14; C12G 3/025; C12R 1/645; C12R 1/865

(52) U.S. Cl. CPC C12G 1/14; C12G 3/025; C12G 2200/05; C12G 2200/11; C12R 2001/645; C12R 2001/865

(57) ABSTRACT

The invention relates to beverages, and to low or substantially no alcohol beverages, i.e. ethanol-reduced or ethanol-free beverages. In particular, the invention relates to substantially ethanol-reduced or ethanol-free wines, and to ethanol-reduced or ethanol-free juice-based beverages, and to methods for making such beverages. The invention also extends to fermentation processes for producing no or low alcohol beverages and to the optimisation of such processes.

The present invention relates to beverages, and particularly, although not exclusively, to low or substantially no alcohol beverages, i.e. ethanol-reduced or ethanol-free beverages. In particular, the invention relates to substantially ethanol-reduced or ethanol-free wines, and to ethanol-reduced or ethanol-free juice-based beverages, and to methods for making such beverages. The invention also extends to fermentation processes for producing no or low alcohol beverages and to the optimisation of such processes.

The global consumption of non-alcoholic beverages, such as wines, is on the rise. Consumers demand daily consumption of healthier and more functional beverages. Multiple health benefits have been attributed to the antioxidant polyphenol compounds found in wine, for instance. These include the ability of these antioxidants to reduce the risk of multiple diseases, including certain cancers and cardiovascular diseases, enhance brain functions, lower bad cholesterol, and regulate blood sugar.

In contrast to traditional soft beverages, wine is subject to consumption restrictions due to its higher alcohol content. Despite the abundance of research on the subject and the health benefits associated with moderate wine consumption, many cardiologists do not recommend consuming more wine due to the effects and risks of alcohol. Therefore, there is a need for beverages that provide the health benefits of wine without the negative effects of alcohol. Thus, there has been an increase in demand for natural and healthy beverages with zero to very low levels of alcohol (less than 0.5% ABV) and low levels of sugar.

Beverages containing no and low alcohol concentrations are labelled in accordance with local legal requirements and regulations. In Europe, the United States and Australasia, beverages with less than 0.5% in ABV (percentage (%) of alcohol per volume) are considered “non-alcoholic”. Low alcohol drinks in the United Kingdom must contain less than 1.2% ABV, while the term “alcohol-free” should only be applied to beverages with an ABV of less than 0.05% (according to the 2018 guidelines).

Fermentation of beverages is a well-known industrial process for producing flavourful beverages, such as wine, beer, and cider by using yeast (naturally present or added) to convert simple carbohydrates into various metabolites, including alcohol and carbon dioxide. The fermentation of saccharine solutions of plant infusions, such as sugar solutions of tea leaf infusions, can also be used to create distinctive flavours.

Kombucha, for example, is created by the fermentation of a sugary solution of tea in the presence of a symbiotic culture of yeast and bacteria producing beverages that contain acetic acid (or ethanal) due to the conversion/oxidation of ethanol into acetic acid, and therefore, contains low or very low level of ethanol, but has a vinegary taste that can be polarising for consumer.

Partial fermentation of low sugary infusions has been used and described to produce beverages with low alcohol content by stopping the fermentation early on, or by converting low sugary infused solutions.

De-alcoholisation, a general term for the physical process of removing alcohol from a liquid, is used to produce the majority of alcohol-free or non-alcoholic wines presently available on the market. Wine can be de-alcoholised using a variety of techniques, including distillation (including vacuum distillation under reduced atmospheric pressure), spinning cone distillation/fractionation, and de-alcoholisation via an osmotic membrane. Unfortunately, during the process of removing the alcohol, many other volatile compounds and ingredients are also removed, resulting in a de-alcoholised wine that lacks some of the healthy compounds and flavours associated with traditional wine, and therefore has a significantly altered flavour and reduced mouthfeel associated with traditional wine.

In addition, traditional alcohol-free or non-alcoholic wines are typically high in polyphenols, acids, and high molecular weight sulphur compounds, which impart astringent, acidic, rubber, sewage, spoiled eggs, pungent, or cooked oil flavours to the wine.

The inventor has made the surprisingly discovery that it is possible to ferment juices, solutions of juices or concentrated juices, or to referment previously fermented drinks, such as de-alcoholised wine (sometimes with added juices or concentrated juices) using a combination of different yeast species, to produce de-alcoholised wine with no or very low levels of alcohol (i.e. less than 0.5% ABV) while maintaining the standard characteristics, body, flavour, aroma and mouthfeel of traditional alcoholic wine, or to produce other non-alcoholic beverages with no or very low levels of alcohol (i.e. less than 1.2% ABV). The inventor has used various yeast strains, such as Saccharomyces cerevisiae yeasts, through alternate cycles of aerobic and anaerobic fermentation, to produce these different ethanol-reduced or ethanol-free beverages.

Thus, in a first aspect of the invention, there is provided a method for producing a de-alcoholised wine, the method comprising re-fermenting a reaction mixture comprising a previously de-alcoholised wine with one or more yeasts, thereby producing the de-alcoholised wine.

It will be appreciated that unlike regular, full-strength (in terms of alcohol) wine, a de-alcoholised wine has a reduced alcohol content. A de-alcoholised wine is typically obtained by removing the alcohol content of a regular, full-strength wine with industrial processes, such as distillation, including vacuum distillation (under reduced atmospheric pressure), spinning cone distillation/fractionation, and de-alcoholisation via an osmotic membrane. It will be understood that, unlike the standard de-alcoholisation process used to obtain traditional de-alcoholised wines, the re-fermentation used in the present invention involves adding a yeast to a previously fermented product (such as a previously de-alcoholised wine), sometimes with a fermentable sugar, to initiate a second fermentation of the product. Thus, the product may be described as being “twice-fermented”.

The starting material (i.e., the previously de-alcoholised wine) can be a previously de-alcoholised red wine, previously de-alcoholised rosé, or previously de-alcoholised white wine, with varying levels of residual alcohol, such as alcohol-free wines (for wines with less than 0.05% ABV) or non-alcoholic wine (for wines with less than 0.5% ABV). Various methodologies, including gas chromatography, the standard method, can be used to determine alcohol content expressed as a percentage (%) ABV. However, alcohol detectors, such as those manufactured by Rare Combination LLC, are more practical for day-to-day operations.

The starting material (i.e., the previously de-alcoholised wine) makes up the largest portion of the re-fermentation reaction mixture. Preferably, the previously de-alcoholised wine represents between 70% and 98% (v/v) of the reaction mixture, between 80% and 98% (v/v) of the reaction mixture, between 85% and 98% (v/v) of the reaction mixture, or more preferably 85% and 95% (v/v) of the reaction mixture. In a preferred embodiment, the previously de-alcoholised wine is 90% or 95% (v/v) of the reaction mixture.

In one embodiment, the reaction mixture further comprises one or more fruit juices or one or more fruit juice concentrates. Preferably, the fruit is selected from grape, but other fruit juices can be used, such as apple, pear, blackcurrant, berry, and/or mango. In another embodiment, the fruit juice may comprise grape must. Preferably, the grape must can be microfiltered or pasteurised. Different grape varieties have been tested with consistent results regardless of grape type, origin, or variety.

The one or more fruit juices may be used in their pure form or diluted. Additionally, the one or more fruit juices may be obtained by diluting concentrated juice to the desired sugar level.

Advantageously, the one or more fruit juice provides the sugar(s) necessary for yeast nutrition during fermentation. Additionally, the juice also imparts distinct fruity aromas and flavours to the resulting de-alcoholised wine.

In one embodiment, the one or more fruit juice is provided in a concentration of less than 20% (v/v) of the reaction mixture, less than 15% (v/v) of the reaction mixture, preferably at a concentration of less than 12% (v/v) of the reaction mixture, and more preferably at a concentration of less than 10% (v/v) of the reaction mixture.

In yet another embodiment, the reaction mixture preferably further comprises granular sugar, simple sugar or monosaccharides, such as glucose, fructose and galactose, and/or disaccharides, such as sucrose.

It will be appreciated that fruit juices naturally contain sugar levels that are dependent on the nature of the fruit and their amount in the reaction mixture (i.e., the solution comprising the previously de-alcoholised wine and the different additives). Addition of concentrated juices can also increase the total sugar content before fermentation, as well as the addition of granular sugar or sucrose. The sugar content can be expressed in percentage (%), in grams per litre (g/L), or can be measured by refractometer using the Brix scale.

Brix scale is used to measure the total amount of dissolved solids in a liquid, and is commonly employed to determine the amount of dissolved sugar in an aqueous solution. The measurement is typically performed by refractometers and is expressed in degrees Brix or ° Br, where 1°Br corresponds to one gram of sugar in 100 ml of distilled water, measured at 20° C.

The Brix level of the reaction mixture before re-fermentation may be typically between 2 and 15° Br, preferably between 2.5 and 12° Br, or between 3 and 10° Br, and more preferably, between 3 and 8° Br.

The Brix levels of concentrated fruit juices range from 50 to 90° Br. Therefore, they are typically diluted to Brix levels of at least 2° Br, preferably between 2.5 and 12° Br, or between 3 and 10° Br, and more preferably between 3 and 8° Br, which provide optimal fermentation conditions for the yeast.

It will be appreciated that by adding concentrated fruit juices and/or sugar or diluting concentrated fruit juices, the sugar content of the reaction mixture will be adjusted to the desired level. For example, mixing 2% to 5% of a grape juice concentrate at 60 to 65° Br with 1% to 5% of apple juice concentrate or pear juice concentrate at 65 to 70° Br, and optionally 0.3% to 2% of sucrose, produces a juice solution of between 3 and 8° Br.

Preferably, the total sugar content in the reaction mixture is less than 80 g/L. Preferably, the total sugar content in the reaction mixture is between 20 g/L and 80 g/L, between 20 g/L and 70 g/L, between 20 g/L and 60 g/L, between 20 g/L and 50 g/L, between 20 g/L and 40 g/L, or between 20 g/L and 30 g/L. Preferably, the total sugar content in the reaction mixture is between 30 g/L and 80 g/L, between 30 g/L and 70 g/L, between 30 g/L and 60 g/L, between 30 g/L and 50 g/L, or between 30 g/L and 40 g/L.

Typically, the total sugar content in the reaction mixture is between 10 g/L and 80 g/L, between 10 g/L and 70 g/L, between 10 g/L and 60 g/L, between 10 g/L and 50 g/L, between 10 g/L and 40 g/L, or between 10 g/L and 30 g/L.

In embodiments in which the total sugar content in the reaction mixture is within the ranges described above, the resulting re-fermented de-alcoholised wine preferably has a residual sugar (i.e., the sugar per litre remaining after fermentation) of 20 g/L or less, 18 g/L or less, 16 g/L or less, 14 g/L or less, 12 g/L or less, 10 g/L or less, 8 g/L or less, 6 g/L or less, 4 g/L or less, or 2 g/L or less. Sugar is indispensable in wine making because, in addition to providing substrate for the yeast during fermentation, the residual sugar in the resulting product enables the classification of the wine in dry, dry-medium, medium sweet, or sweet wine. Therefore, a wine with less than 2 g/L is considered “bone dry”, whereas a wine with more than 45 g/L is considered “sweet”. The re-fermented de-alcoholised wines produced by the methods of the present invention preferably range from “bone dry” to “medium sweet”.

In one embodiment, the reaction mixture further comprises tea extract, catechin and/or polyphenols. The tea extract (preferably, green tea extract) may be a powdered tea extract or a liquid tea extract. Cathechins and polyphenols may be provided in powdered or liquid forms. The addition of tea extract (as a source of flavonoid-type polyphenols), catechin, or polyphenols improves the mouthfeel and body of the wine without deleteriously altering the fermentation process.

Preferably, tea extract, catechin and/or polyphenols are independently provided at a concentration of between 0.25 g/L and 2 g/L, and more preferably between 0.5 g/L and 1 g/L.

In another embodiment, preferably the reaction mixture further comprises copper or ions of copper (Cu²⁺), usually in the form of copper citrate or copper sulphate. Copper is used to offset the sulphuric or off-odours and as a treatment to remove ‘reductive’ aromas during the fermentation and can act as a mediator of reactions between dissolved oxygen and wine components, such as phenolic compounds and ascorbic acid.

Preferably, the copper is provided at a concentration of less than 1 mg/L, more preferably less than 0.5 mg/L. In a preferred embodiment, the copper is provided at a concentration of about 0.4 mg/L.

In one embodiment, the one or more yeasts used to re-ferment the reaction mixture comprising the previously de-alcoholised wine is from a genus of yeast selected from a group of yeast genuses consisting of: Saccharomyces, Torulaspora, Lachancea, Metschnikowia, and Schizosaccharomyces, or a combination thereof.

In one embodiment, the one or more yeasts used to re-ferment the reaction mixture comprises a yeast of the genus, Saccharomyces. Preferably, the Saccharomyces is S. cerevisiae.

Advantageously, the inventors have used a recombinant S. cerevisiae that has been specifically engineered to reduce the concentration of alcohol produced during the re-fermentation process to less than 0.5% ABV. The recombinant strain is Saccharomyces cerevisiae strain BR 23007 deposited under DSM 34577 on 5^{th} of April 2023 at the Institute Deutsche Sammlung von Mikroorganismen und Zellkulturen GmbH (DSMZ), and is referred to as DSM34577 or Saccharomyces cerevisiae strain 322.10.4.

By targeting specific amino acids (by punctual mutation) in the genome of S. cerevisiae using CRISPR or similar technologies, S. cerevisiae strain DSM34577 exhibits a significant decrease in ethanol production by altering the expression level of numerous genes and functions in the central metabolic pathways, as follows:
Reducing the glucose uptake by attenuating glycolysis flux and enhancing the TCA cycle;
Enhancing histidine and purine synthesis and ATP synthesis; and
Ultimately resulting in disturbed Crabtree effect (due to reduced sugar uptake), higher biomass yield, higher CO2 release and ultimately less ethanol production.

The low-ethanol producing S. cerevisiae strain DSM34577 is characterised in that it expresses a mutated SPT-15 gene. More specifically, the mutated SPT-15 gene encodes an amino acid sequence comprising mutations at at least three positions selected from: I46, D56, S118, Y195, and L205 in the amino acid sequence of the wild-type S. cerevisiae SPT-15 gene. The mutations at positions I46, D56, S118, Y195, and L205 are I46M, D56G, S118P, Y195H, and L205S, respectively. The amino acid sequence of the mutated SPT-15 gene is represented herein as SEQ ID No: 1, as follows:

[SEQ ID No: 1]
MADEERLKEFKEANKIVFDPNTRQVWENQNRDGTKPATTFQSEEDMKRAA
PESEKGTSATSGIVPTLQNIVATVTLGCRLDLKTVALHARNAEYNPKRFA
AVIMRIREPKTTALIFAPGKMVVTGAKSEDDSKLASRKYARIIQKIGFAA
KFTDFKIQNIVGSCDVKFPIRLEGLAFSHGTFSSYEPELFPGLIHRMVKP
KIVLSIFVSGKIVLTGAKQREEIYQAFEAIYPVLSEFRKM

Furthermore, the S. cerevisiae strain DSM34577 comprises at least one genomic copy of the mutated SPT-15 gene, and the at least one copy of the mutant SPT-15 gene is inserted into the genomic ADH-1 gene locus which functionally deletes or disrupts the open reading frame of the ADH-1 gene.

Advantageously, the transcription factor TATA-binding Protein (TBP) encoded by the wild-type S. cerevisiae SPT-15 gene regulates the transcription of numerous genes in yeasts. Surprisingly, the expression of at least one genomic copy of the mutated SPT-15 gene in S. cerevisiae, stably reduces ethanol production during fermentation not only in the recombinant strain, but also in subsequent generations.

Preferably, therefore, the previously de-alcoholised wine is re-fermented with a genetically engineered yeast strain comprising a mutated SPT-15 gene, or a homologue or paralogue thereof. In one embodiment, the yeast strain comprising the mutated SPT-15 gene is of the genus selected from the group consisting of: Saccharomyces spp., Kazachstani spp., Torulaspora spp, and Pichia spp. Preferably, the yeast strain comprising the mutated SPT-15 gene is of the genus Saccharomyces spp. Preferably, the yeast strain comprising the mutated SPT-15 gene is S. cerevisiae or S. pastorianus.

Preferably, the mutated SPT-15 gene, or a homologue or paralogue thereof, comprises mutations at at least three positions selected from: I46, D56, S118, Y195, and L205 in the amino acid sequence of the wild-type S. cerevisiae SPT15 gene. Preferably, the mutated SPT-15 gene, or a homologue or paralogue thereof, comprises mutations at at least four, or most preferably, five positions, selected from: I46, D56, S118, Y195, and L205 in the amino acid sequence of the wild-type S. cerevisiae SPT15 gene. Preferably, the mutated SPT15 gene, or a homologue or paralogue thereof, comprises the mutations: I46M, D56G, S118P, Y195H, and L205S. Preferably, the mutated SPT-15 gene encodes an amino acid sequence substantially as set out in SEQ ID No: 1.

Preferably, the previously de-alcoholised wine is re-fermented with a genetically engineered yeast strain comprising at least one genomic copy of the mutated SPT-15 gene, or a homologue or paralogue thereof. Preferably, the at least one copy of the mutant SPT-15 gene is inserted into the genomic ADH-1 gene locus which functionally deletes or disrupts the open reading frame of the ADH-1 gene. Accordingly, in a preferred embodiment, the one or more yeasts used to re-ferment the reaction mixture is S. cerevisiae strain DSM34577.

Preferably, the yeast strain is between 10⁸ and 10⁹ cfu in the reaction mixture. Preferably, the S. cerevisiae strain DSM34577 is used in the reaction mixture at a concentration of 3 ml/L, 2 ml/L or 1 ml/L.

In another embodiment, the one or more yeasts used to re-ferment the reaction mixture is a wild-type of S. cerevisiae (referred to as “HF 13011”), which is the wild-type progenitor of strain DSM34577. This strain is commercially available from Weiss Biotech GmbH, 59387 Ascheberg, Germany.

In yet another embodiment, the one or more yeasts used to re-ferment the reaction mixture is wild type of S. cerevisiae (referred to as HF 13309). This strain is commercially available from Weiss Biotech GmbH, 59387 Ascheberg, Germany.

In one embodiment, the one or more yeasts used to re-ferment the reaction mixture comprises a yeast of the genus, Toluraspora. Preferably, the Torulaspora is T. delbrueckii, more preferably T. delbrueckii wildtype wine yeast (referred to as HF 13015). This strain is commercially available from Weiss Biotech GmbH, 59387 Ascheberg, Germany.

When the one or more yeasts is provided in powder form (i.e. powder biomass), the one or more yeasts is hydrated for at least 5 minutes, at least 10 minutes, at least 15 minutes, at least 20 minutes, at least 25 minutes or at least 30 minutes prior to inoculating the reaction mixture. Preferably, the one or more yeasts is hydrated for 30 minutes prior to inoculating the reaction mixture.

When the one or more yeasts is provided in liquid form (i.e. liquid biomass), the one or more yeast is not hydrated prior to inoculating the reaction mixture.

It should be appreciated that except for the wild-type S. cerevisiae strain HF 13309, which was provided in liquid form and, therefore, does not require prior hydration, all yeasts used in the present invention were provided in powder form and, therefore, require hydration before fermentation.

In one embodiment of the method, the reaction mixture comprising the previously de-alcoholised wine is inoculated with a single yeast strain or species.

In a preferred embodiment, however, the reaction mixture comprising the previously de-alcoholised wine is inoculated with at least two yeast strains or species.

Preferably, the at least two yeast strains comprise Saccharomyces spp. and Torulaspora spp., more preferably, S. cerevisiae and T. delbrueckii. Preferably, the S. cerevisiae is selected from the wild-type S. cerevisiae strain HF 13309, the wild-type S. cerevisiae strain HF 13011 or more preferably a genetically engineered S. cerevisiae strain 322.10.4 (referred to as DSM34577). Preferably, the Torulaspora spp. is Torulaspora strain HF 13015.

In a most preferred embodiment, the at least two yeasts strains comprise S. cerevisiae DSM34577 and Torulaspora strain HF 13015.

Single inoculation of the genetically engineered S. cerevisiae yeast DSM34577 is also a preferred embodiment.

In one embodiment, the one or more yeasts are co-inoculated i.e., they added to the reaction mixture at the same time.

In another embodiment, the one or more yeasts are sequentially inoculated, i.e., one yeast is added to the reaction mixture first, and the second or more yeast are added subsequently.

In one embodiment, the total amount of yeast inoculated in the reaction mixture is at a concentration of between 0.1 g/L and 5 g/L, between 0.1 g/L and 4 g/L, between 0.1 g/L and 3 g/L, between 0.1 g/L and 2 g/L, between 0.1 g/L and 1 g/L, or between 0.1 g/L and 0.5 g/L. Preferably, the total amount of yeast is between 10⁸ and 10⁹ cfu in the reaction mixture.

Fermentation is typically conducted at controlled temperature ranging from 10° C. to 30° C., for 7 to 15 days or longer, to produce optimal flavour. In addition to the important role played in producing fermented beverages with complex aromas, the temperature of the fermentation also facilitates the production of biomass to ensure and maintain a low level of alcohol after fermentation.

Preferably, the re-fermentation takes place in a temperature-controlled fermenter. Preferably, the re-fermentation takes place at a temperature of between 10° C. and 30° C., more preferably between 11° C. and 29° C., between 13° C. and 27° C., or between 15° C. and 26° C. Preferably, the re-fermentation takes place at a temperature of between 16° C. and 25° C., between 17° C. and 24° C., between 19° C. and 25° C., or between 20° C. and 25° C.

Preferably, fermentation is carried out in fewer than 15 days, more preferably between 2 and 12 days, between 2 and 10 days, between 2 and 8 days, or between 2 and 7 days.

Advantageously, the inventors have shown that combining both aerobic and anaerobic fermentations significantly improves the overall characteristics of the resulting de-alcoholised wine. Thus, preferably, the re-fermentation step of the method comprises aerobic and/or anaerobic re-fermentation, and more preferably, aerobic and anaerobic re-fermentation of the previously de-alcoholised wine. Preferably, the re-fermentation step comprises aerobic fermentation followed by anaerobic re-fermentation of the previously de-alcoholised wine.

Advantageously, a first phase of aerobic fermentation followed by a second phase of anaerobic fermentation optimises the sugar consumption by the different microorganisms (particularly those producing no or very low levels of alcohol i.e., less than 0.5%, such as the S. cerevisiae DSM34577 and Torulaspora strain HF 13015). The initial respiratory (aerobic) phase is preferably conducted for 12 to 120 hours, 12 to 96 hours, 12 to 72 hours, 12 to 48 hours, or 12 to 24 hours. Preferably, aerobic fermentation is conducted for 12 to 72 hours, and more preferably 12 to 48 hours, in order to allow the biomass to develop and reduce the total amount of alcohol produced throughout the process.

Advantageously, the inventor has demonstrated that extending the aerobic phase to up to 72 hours further reduces the alcohol content of the end-product, i.e., the de-alcoholised wine. More preferably, aerobic fermentation is conducted for 48 hours.

Surprisingly, aerobic refermentation of the previously de-alcoholised wine (i.e., the starting material) significantly reduces the impact of negative volatile sulphur compounds, thereby improving the overall sensorial profile of the resulting wine.

By re-fermenting previously de-alcoholised wine, a portion of the negative sulphur compounds are removed by aerobic fermentation, and the remaining negative sulphur compounds are treated with Cu2+ or CuSO4.

During aerobic fermentation, an adequate oxygen level is required to ensure that the biomass is formed, and sugars present in the reaction mixture are consumed while limiting alcohol production.

During the aerobic fermentation, dissolved oxygen content is preferably at least 2.5 mg/L, preferably above 3 mg/L, above 3.5 mg/L, and even more preferably above 4 mg/L. To ensure that this level is maintained during the aerobic fermentation, oxygen levels in the reaction mixture may be increased by injecting additional oxygen in the reaction mixture, for example, by recirculating, or by using a venturi device, or by agitating or vigorously agitating the fermenter. Using a Digital Oxygen Meter, such as those manufactured by the MRC Laboratory Equipment Manufacturer, oxygen content can be measured.

Preferably, the aerobic fermentation is performed by Saccharomyces spp. and Torulaspora spp., more preferably, S. cerevisiae and T. delbrueckii. Preferably, the S. cerevisiae is selected from the wild-type S. cerevisiae strain HF 13309, the wild-type S. cerevisiae strain HF 13011 or more preferably a genetically engineered S. cerevisiae strain 322.10.4 (referred to as DSM34577). Preferably, the Torulaspora spp. is Torulaspora strain HF 13015.

In another embodiment, aerobic fermentation is followed by anaerobic fermentation.

Preferably, the anaerobic fermentation is performed by Saccharomyces spp. and Torulaspora spp., more preferably, S. cerevisiae and T. delbrueckii. Preferably, the S. cerevisiae is selected from the wild-type S. cerevisiae strain HF 13309, the wild-type S. cerevisiae strain HF 13011 or more preferably a genetically engineered S. cerevisiae strain 322.10.4 (referred to as DSM34577). Preferably, the Torulaspora spp. is Torulaspora strain HF 13015.

Preferably, anaerobic fermentation is conducted for 12 to 72 hours, 12 to 48 hours, or 12 to 24 hours. Preferably, anaerobic fermentation is conducted for 12 to 48 hours. More preferably, anaerobic fermentation is conducted for 48 hours.

Preferably, anaerobic fermentation is carried out at a temperature of between 10° C. and 30° C., more preferably between 11° C. and 29° C., between 13° C. and 27° C., or between 15° C. and 26° C. Preferably, the re-fermentation takes place at a temperature of between 16° C. and 25° C., between 17° C. and 24° C., between 19° C. and 25° C., or between 20° C. and 25° C.

In a preferred embodiment, anaerobic fermentation takes place at a temperature of between 22° C. and 25° C.

In a preferred embodiment, the method of the first aspect comprises refermenting aerobically and/or anaerobically, a previously de-alcoholised wine with S. cerevisiae, preferably S. cerevisiae comprising a mutated SPT-15 gene, or a homologue or paralogue thereof, thereby producing a de-alcoholised wine comprising less than 1% ABV.

In a most preferred embodiment, the method of the first aspect comprises refermenting aerobically and/or anaerobically, a previously de-alcoholised wine with S. cerevisiae, preferably S. cerevisiae comprising a mutated SPT-15 gene, or a homologue or paralogue thereof, thereby producing a de-alcoholised wine comprising less than 0.5% ABV.

In a preferred embodiment, the method of the first aspect comprises refermenting aerobically and/or anaerobically, a previously de-alcoholised wine with S. cerevisiae, preferably S. cerevisiae comprising a mutated SPT-15 gene, or a homologue or paralogue thereof, and T. delbrueckii, thereby producing a de-alcoholised wine comprising less than 1% ABV.

In a most preferred embodiment, the method of the first aspect comprises refermenting aerobically and anaerobically, a previously dealcoholised wine with S. cerevisiae, preferably S. cerevisiae comprising a mutated SPT-15 gene, or a homologue or paralogue thereof, and T. delbrueckii, thereby producing a dealcoholised wine comprising less than 0.5% ABV.

In a preferred embodiment, the method of the first aspect comprises refermenting a reaction mixture comprising 90% (v/v) of previously de-alcoholised white wine (0.0% ABV) (DAWW), 6.25% (v/v) of must or grape juice pasteurized (MP), 2% (v/v) of grape juice concentrate (GJC), and 1 ml/L S. cerevisiae DSM34577 and 0.4 mg/L of Cu²⁺.

In a second aspect, there is provided a de-alcoholised wine obtained, or obtainable, by the method of the first aspect.

Preferably, the de-alcoholised wine of the second aspect further comprises sugar, natural flavour, natural sweetener (such as stevia), colouring agent, preservative, and/or vitamins added after the re-fermentation.

Advantageously, adding sugar to the de-alcoholised wine obtained by the method of the first aspect after fermentation, but before bottling the wine, further reduces acidity and bitterness.

In one embodiment, the de-alcoholised wine of the second aspect is a low-alcohol de-alcoholised wine (i.e., comprises up to no more than 1.2% ABV). In one embodiment, the de-alcoholised wine of the second aspect comprises up to 1.2% ABV, preferably less than 1.2% ABV, less than 1.1% ABV, or less than 1.0% ABV. In a preferred embodiment, the de-alcoholised wine comprises less than 0.9% ABV, preferably less than 0.8% ABV, or less than 0.7% ABV. In a more preferred embodiment, the de-alcoholised wine comprises less than 0.6% ABV or less than 0.5% ABV.

In another embodiment, the de-alcoholised wine of the second aspect is a non-alcoholic de-alcoholised wine (i.e., comprises up to no more than 0.5% ABV).

In yet another embodiment, the de-alcoholised wine of the second aspect is an alcohol-free de-alcoholised wine (i.e., comprises up to 0.05% ABV). Thus, preferably the de-alcoholised wine comprises less than 0.4% ABV, less than 0.3% ABV, less than 0.2% ABV, and more preferably, less than 0.1% ABV.

Preferably, the de-alcoholised wine of the second aspect, has a pH between 3.0 and 5.0, between 3.0 and 4.5, between 3.0 and 4.0, more preferably a pH above 3.0, and less than 4.5.

In one embodiment, the de-alcoholised wine of the second aspect is a white wine, red wine, or rose wine. In one embodiment, the de-alcoholised wine of the second aspect is a carbonated or sparkling wine, or a still or non-carbonated wine.

Carbonation of the de-alcoholised wine is a normal by-product of fermentation. In a preferred embodiment, the de-alcoholised wine is carbonated by mechanical injection of carbon dioxide, which produces high pressure in the wine and results in the formation of effervescent bubbles.

In a third aspect of the invention, there is provided a method for producing a reduced alcohol or non-alcoholic beverage, the method comprising fermenting a reaction mixture comprising one or more fruit juices, or one or more fruit juice concentrates, with one or more yeasts, wherein the one or more yeasts comprises a genetically engineered yeast strain comprising a modified SPT-15 gene or a homologue or paralogue thereof, or wild-type Saccharomyces cerevisiae strain HF 13309, to thereby produce a reduced alcohol or non-alcoholic beverage.

According to European Union regulations, a non-alcoholic beverage must contain a maximum of 1.2% ABV (as defined above). Beverages containing more than 1.2% ABV must disclose the alcoholic strength of the beverage on their labels.

Preferably, therefore, the one or more fruit juice or fruit just concentrate is fermented with a genetically engineered yeast strain comprising a mutated SPT-15 gene, or a homologue or paralogue thereof. Preferably, the mutated SPT-15 gene, or a homologue or paralogue thereof, comprises mutations at at least three positions selected from: I46, D56, S118, Y195, and L205 in the amino acid sequence of the wild-type S. cerevisiae SPT15 gene. Preferably, the mutated SPT-15 gene, or a homologue or paralogue thereof, comprises mutations at at least four or five positions selected from: I46, D56, S118, Y195, and L205 in the amino acid sequence of the wild-type S. cerevisiae SPT15 gene.

Preferably, the mutated SPT15 gene, or a homologue or paralogue thereof, comprises the mutations: I46M, D56G, S118P, Y195H, and L205S. Preferably, the one or more fruit juice or fruit just concentrate is fermented with a genetically engineered yeast strain comprising at least one genomic copy of the mutated SPT-15 gene, or a homologue or paralogue thereof. Preferably, the at least one copy of the mutant SPT-15 gene is inserted into the genomic ADH-1 gene locus which functionally deletes or disrupts the open reading frame of the ADH-1 gene.

In one embodiment, the yeast strain comprising the mutated SPT-15 gene is of the genus selected from the group consisting of: Saccharomyces spp., Kazachstani spp., Torulaspora spp, and Pichia spp. Preferably, the yeast strain comprising the mutated SPT-15 gene is of the genus Saccharomyces spp. Preferably, the yeast strain comprising the mutated SPT-15 gene is S. cerevisiae or S. pastorianus.

Preferably, the one or more yeasts used to ferment the reaction mixture comprising the one or more fruit juice or fruit juice concentrate further comprises yeast selected from a group of yeast genuses consisting of: Saccharomyces, Torulaspora, Lachancea, Metschnikowia, and Schizosaccharomyces, or a combination thereof. Preferably, the one or more yeasts used to ferment the reaction mixture comprising the one or more fruit juice or concentrate further comprises Saccharomyces, preferably S. cerevisae.

Accordingly, in a preferred embodiment, the one or more yeasts used to ferment the reaction mixture is S. cerevisiae strain DSM34577.

In another embodiment, the one or more yeasts used to ferment the reaction mixture is S. cerevisiae strain HF 13309.

In one embodiment, the one or more yeasts used to ferment the reaction mixture further comprises a wild-type of S. cerevisiae HF 13011.

In one embodiment, the one or more yeasts used to ferment the reaction mixture further comprises a yeast of the genus, Toluraspora. Preferably, the Torulaspora is T. delbrueckii, more preferably T. delbrueckii wildtype wine yeast HF 13015).

In one embodiment of the method, the reaction mixture comprising the one or more fruit juice or fruit just concentrate is inoculated with a single yeast strain or species.

In a preferred embodiment, however, the reaction mixture the one or more fruit juice or fruit just concentrate is inoculated with at least two yeast strains or species.

Preferably, the at least two yeast strains comprise Saccharomyces and Torulaspora, more preferably, S. cerevisiae and T. delbrueckii. Preferably, the S. cerevisiae is selected the wild-type S. cerevisiae strain HF 13309, the wild-type S. cerevisiae strain HF 13011 or more preferably a genetically engineered S. cerevisiae strain 322.10.4 (referred to as DSM34577). Preferably, the Torulaspora is Torulaspora strain HF 13015.

In a most preferred embodiment, the at least two yeasts strains comprise S. cerevisiae DSM34577 and Torulaspora strain HF 13015.

Single inoculation of the genetically engineered S. cerevisiae yeast DSM34577 is also a preferred embodiment.

In one embodiment, the one or more yeasts are co-inoculated i.e., they added to the reaction mixture at the same time.

In another embodiment, the one or more yeasts are sequentially inoculated, i.e., one yeast is added to the reaction mixture first, and the second or more yeast are added subsequently.

In one embodiment, the total amount of yeast inoculated in the reaction mixture is at a concentration of between 0.1 g/L and 5 g/L, between 0.1 g/L and 4 g/L, between 0.1 g/L and 3 g/L, between 0.1 g/L and 2 g/L, between 0.1 g/L and 1 g/L, or between 0.1 g/L and 0.5 g/L. Preferably, the total amount of yeast is between 10⁸ and 10⁹ cfu in the reaction mixture.

The starting material (i.e., the one or more fruit juices, or one or more fruit juice concentrates) makes up less than 20% (v/v) of the fermentation reaction mixture. Preferably, the fruit juice is provided in a concentration of less than 15% (v/v) of the reaction mixture, preferably at a concentration of less than 12% (v/v) of the reaction mixture, and more preferably at a concentration of less than 10% (v/v) of the reaction mixture.

In one embodiment, the fruit is selected from grape, but other fruit juices can be used, such as, apple, pear, blackcurrant, berry, and/or mango. In another embodiment, the fruit juice may comprise grape must. Preferably, the grape must can be microfiltered or pasteurised. Different grape varieties have been tested with consistent results regardless of grape type, origin, or variety.

The one or more fruit juices may be used in their pure form or diluted. Additionally, the one or more fruit juices may be obtained by diluting concentrated juice to the desired sugar level.

The fruit juices provide the sugars necessary for yeast nutrition during fermentation. Additionally, the juices also impart distinct fruity aromas and flavours to the resulting non-alcoholic beverage. In one embodiment, the reaction mixture further comprises granular sugar, simple sugar or monosaccharides such as glucose, fructose and galactose, and/or disaccharides such as sucrose. It will be appreciated that fruit juices naturally contain sugar levels that are dependent on the nature of the fruit and their amount in the reaction mixture. Addition of sugar also increases the total sugar content before fermentation.

The Brix level of the reaction mixture before fermentation is typically between 2 and 15° Br, preferably between 2.5 and 12° Br, 3 and 10° Br, and more preferably between 3 and 8° Br. The brix levels of concentrated fruit juices ranges from 50 to 90° Br. Therefore, there are typically diluted to brix levels of at least 2°Br, preferably between 2.5 and 12° Br, 3 and 10° Br, and more preferably between 3 and 8° Br, which provide optimal fermentation conditions for the yeast.

It will be appreciated concentrated fruit juices and sugar or diluted concentrated fruit juices will adjust the sugar content of the reaction mixture to the desired level. For example, mixing 22% to 5% of a grape juice concentrate at 60 to 65° Br with 1% to 5% of apple juice concentrate or pear juice concentrate at 65 to 70° Br, and optionally 0.3% to 2% of sucrose produces a juice solution between 3 and 8° Br.

Preferably, the total sugar content in the reaction mixture is less than 80 g/L. Preferably, the total sugar content in the reaction mixture is between 20 g/L and 80 g/L, between 20 g/L and 70 g/L, between 20 g/L and 60 g/L, between 20 g/L and 50 g/L, between 20 g/L and 40 g/L, or between 20 g/L and 30 g/L. Preferably, the total sugar content in the reaction mixture is between 30 g/L and 80 g/L, between 30 g/L and 70 g/L, between 30 g/L and 60 g/L, between 30 g/L and 50 g/L, or between 30 g/L and 40 g/L.

In embodiments in which the total sugar content in the reaction mixture is within the range described above, the resulting non-alcoholic beverage preferably has a residual sugar (i.e., the sugar per litre remaining after fermentation) of 20 g/L or less, 18 g/L or less, 16 g/L or less, 14 g/L or less, 12 g/L or less, 10 g/L or less, 8 g/L or less, 6 g/L or less, 4 g/L or less, or 2 g/L or less.

In one embodiment, the reaction mixture further comprises tea extract, catechin and/or polyphenols. The tea extract, preferably, green tea extract may be a powdered tea extract or a liquid tea extract. Cathechins and polyphenol may be provided in powdered or liquid forms. Preferably, tea extract, catechin and/or polyphenols are provided at a concentration of between 0.25 g/L and 2 g/L, and more preferably, between 0.5 g/L and 1 g/L.

Fermentation is typically conducted at controlled temperature ranging from 10° C. to 30° C., for 7 to 15 days or longer to produce optimal flavour. In addition to the important role played in producing fermented beverages with complex aromas, the temperature of the fermentation also facilitates the production of biomass to ensure and maintain a low level of alcohol after fermentation.

Preferably, the fermentation takes place in a temperature-controlled fermenter. Preferably takes place at a temperature of between 10° C. and 30° C., more preferably between 11° C. and 29° C., between 13° C. and 27° C. or between 15° C. and 26° C. Preferably, the fermentation takes place at a temperature of between 16° C. and 25° C., between 17° C. and 24° C., between 19° C. and 25° C. or between 20° C. and 25° C.

Preferably, fermentation is carried out in fewer than 15 days, more preferably between 2 and 12 days, 2 and 10 days, 2 and 8 days, or 2 and 7 days.

Advantageously, the inventor has shown that combining both aerobic and anaerobic fermentations significantly improves the overall characteristics of the resulting non-alcoholic beverage. Thus, preferably, the fermentation step of the method comprises aerobic and/or anaerobic fermentation, and more preferably, aerobic and anaerobic fermentation of the one or more fruit juice or fruit just concentrate.

Preferably, the fermentation step comprises aerobic followed by anaerobic fermentation of the one or more fruit juice or fruit just concentrate.

Advantageously, a first phase of aerobic fermentation followed by a second phase of anaerobic fermentation optimises the sugar consumption by the different microorganisms (particularly those producing no or very low levels of alcohol i.e., less than 0.5%, such as the S. cerevisiae DSM34577 and Torulaspora strain HF 13015). The initial respiratory (aerobic) phase is preferably conducted for 12 to 120 hours, 12 to 96 hours, 12 to 72 hours, 12 to 48 hours, or 12 to 24 hours.

Preferably, aerobic fermentation is conducted for 12 to 72 hours, and more preferably 12 to 48 hours, in order to allow the biomass to develop and reduce the total amount of alcohol produced throughout the process.

Advantageously, the inventor has demonstrated that extending the aerobic phase to up to 72 hours further reduces the alcohol content of the end-product, i.e. the non-alcoholic beverage.

During the aerobic fermentation, dissolved oxygen content is preferably at least 2.5 mg/L, preferably above 3 mg/L, above 3.5 mg/L and even more preferably above 4 mg/L. To ensure that this level is maintained during the aerobic fermentation, oxygen levels in the reaction mixture may be increased by injecting additional oxygen in the reaction mixture, for example, a venturi device or by agitating or vigorously agitating the fermenter. Using a Digital Oxygen Meter, such as those manufactured by the MRC Laboratory Equipment Manufacturer, oxygen content can be measured.

During aerobic fermentation, an adequate oxygen level is required to ensure that the biomass is formed, and sugars present in the reaction mixture are consumed while limiting alcohol production.

In another embodiment, aerobic fermentation is followed by anaerobic fermentation. Preferably, the anaerobic fermentation is performed by Saccharomyces ssp, and/or Torulaspora spp.

Preferably, anaerobic fermentation is conducted for 12 to 72 hours, 12 to 48 hours, or 12 to 24 hours. Preferably, anaerobic fermentation is conducted for 12 to 48 hours. More preferably, anaerobic fermentation is conducted for 48 hours.

Preferably, anaerobic fermentation is carried out at a temperature of between 10° C. and 30° C., more preferably between 11° C. and 29° C., between 13° C. and 27° C., or between 15° C. and 26° C. Preferably, the re-fermentation takes place at a temperature of between 16° C. and 25° C., between 17° C. and 24° C., between 19° C. and 25° C., or between 20° C. and 25° C.

In a preferred embodiment, anaerobic fermentation takes place at a temperature of between 22° C. and 25° C.

In a preferred embodiment, the method of the third aspect comprises fermenting aerobically and/or anaerobically, one or more fruit juice or fruit just concentrate with S. cerevisiae, preferably S. cerevisiae comprising a mutated SPT-15 gene, or a homologue or paralogue thereof, or wild-type S. cerevisiae 13309, thereby producing a non-alcoholic beverage comprising less than 1.2% ABV.

In a most preferred embodiment, the method of the third aspect comprises fermenting aerobically and/or anaerobically, one or more fruit juice or fruit just concentrate with S. cerevisiae comprising a mutated SPT-15 gene, or a homologue or paralogue thereof, or wild-type S. cerevisiae 13309, thereby producing a non-alcoholic beverage comprising less than 0.5% ABV.

In a preferred embodiment, the method of the third aspect comprises fermenting aerobically and/or anaerobically, one or more fruit juice or fruit just concentrate with S. cerevisiae comprising a mutated SPT-15 gene, or a homologue or paralogue thereof, or wild-type S. cerevisiae 13309, and T. delbrueckii, thereby producing a non-alcoholic beverage comprising less than 1.2% ABV.

In a most preferred embodiment, the method of the third aspect comprises fermenting aerobically and/or anaerobically, one or more fruit juice or fruit just concentrate S. cerevisiae comprising a mutated SPT-15 gene, or a homologue or paralogue thereof, or wild-type S. cerevisiae 13309, and T. delbrueckii, thereby producing a non-alcoholic beverage comprising less than 0.5% ABV.

In a preferred embodiment, the method of the third aspect comprises fermenting a reaction mixture comprising 3% of grape juice concentrate (GJC), 0.5% of apple juice concentrate (AJC), 0.75 g/L of green tea extracts (GTEx), 0.3% of granular sugar or sucrose (GS) with 0.4 g/L of Toluraspora HF 13015, and 0.1 ml/L S. cerevisiae HF 13309.

In another preferred embodiment, the method of the third aspect comprises fermenting a reaction mixture comprising 3% of grape juice concentrate (GJC), 0.5% of apple juice concentrate (AJC), 0.75 g/L of green tea extracts (GTEx), 0.3% of granular sugar or sucrose (GS) with 0.4 g/L of Toluraspora HF 13015, and 1 ml/L S. cerevisiae DSM34577.

Preferably, the fermentation is carried out under aerobic conditions between 22° C. and 25° C. for 48 hours.

Preferably, aerobic fermentation is followed by anaerobic fermentation between 22° C. and 25° C. for 48 hours.

In a fourth aspect, there is provided a non-alcoholic beverage obtained, or obtainable, by the method of the third aspect.

Preferably, the non-alcoholic beverage of the fourth aspect, further comprises sugar, natural flavour, natural sweetener (such as stevia), colouring agent, preservative, and/or vitamins added after fermentation.

In one embodiment, the non-alcoholic beverage of the fourth aspect comprises up to 1.2% ABV, preferably less than 1.2% ABV, less than 1.1% ABV, or less than 1.0% ABV. In a preferred embodiment, the de-alcoholised wine comprises less than 0.9% ABV, preferably less than 0.8% ABV, or less than 0.7% ABV. In a more preferred embodiment, the de-alcoholised wine comprises less than 0.6% ABV or less than 0.5% ABV.

Accordingly, in one embodiment, the non-alcoholic beverage of the fourth aspect is a low-alcohol beverage (i.e., comprises up to 1.2% ABV).

In another embodiment, the non-alcoholic beverage of the fourth aspect is a non-alcoholic beverage (i.e., comprises up to 0.5% ABV).

Preferably, the non-alcoholic beverage of the fourth aspect, has a pH between 3.0 and 5.0, between 3.0 and 4.5, between 3.0 and 4.0, more preferably a pH above 3.0 and less than 4.5.

In one embodiment, the non-alcoholic beverage of the fourth aspect is a white non-alcoholic beverage, red non-alcoholic beverage, or rose non-alcoholic beverage.

In one embodiment, the non-alcoholic beverage of the fourth aspect is a carbonated or sparkling non-alcoholic beverage, or a still or non-carbonated non-alcoholic beverage.

Carbonation of the non-alcoholic beverage is a normal by-product of fermentation. In a preferred embodiment, the non-alcoholic beverage is carbonated by mechanical injection of carbon dioxide, which produces high pressure in the non-alcoholic beverage and results in the formation of effervescent bubbles.

In a fifth aspect, there is provided a non-alcoholic beverage comprising the de-alcoholised wine of the second aspect and the non-alcoholic beverage of the fourth aspect.

In one embodiment, the non-alcoholic beverage of the fifth aspect comprises approximately an equal amount (i.e., 50% v/v) of each of the de-alcoholised wine of the second aspect and the non-alcoholic beverage of the fourth aspect.

In another embodiment the non-alcoholic beverage of the fifth aspect comprises 10% (v/v) of the de-alcoholised wine of the second aspect and 90% (v/v) of the non-alcoholic beverage of the fourth aspect, preferably 20% (v/v) of the de-alcoholised wine of the second aspect and 80% (v/v) of the non-alcoholic beverage of the fourth aspect, or 30% (v/v) of the de-alcoholised wine of the second aspect and 70% (v/v) of the non-alcoholic beverage of the fourth aspect, or 40% (v/v) of the de-alcoholised wine of the second aspect and 60% (v/v) of the non-alcoholic beverage of the fourth aspect, or 60% (v/v) of the de-alcoholised wine of the second aspect and 40% (v/v) of the non-alcoholic beverage of the fourth aspect, or 70% (v/v) of the de-alcoholised wine of the second aspect and 30% (v/v) of the non-alcoholic beverage of the fourth aspect, or 80% (v/v) of the de-alcoholised wine of the second aspect and 20% (v/v) of the non-alcoholic beverage of the fourth aspect, or 90% (v/v) of the de-alcoholised wine of the second aspect and 10% (v/v) of the non-alcoholic beverage of the fourth aspect.

Most preferably, the non-alcoholic beverage of the fifth aspect comprises 90% (v/v) of the de-alcoholised wine of the second aspect and 10% (v/v) of the non-alcoholic beverage of the fourth aspect.

Preferably, the non-alcoholic beverage of the fifth aspect, further comprises sugar, natural flavour, natural sweetener (such as stevia), colouring agent, preservative, and/or vitamins added after fermentation.

In one embodiment, the non-alcoholic beverage of the fifth aspect comprises up to 1.2% ABV, preferably less than 1.2% ABV, less than 1.1% ABV, or less than 1.0% ABV. In a preferred embodiment, the de-alcoholised wine comprises less than 0.9% ABV, preferably less than 0.8% ABV, or less than 0.7% ABV. In a more preferred embodiment, the de-alcoholised wine comprises less than 0.6% ABV or less than 0.5% ABV.

Accordingly, in one embodiment, the non-alcoholic beverage of the fifth aspect is a low-alcohol beverage (i.e., comprises up to 1.2% ABV).

In another embodiment, the non-alcoholic beverage of the fifth aspect is a non-alcoholic beverage (i.e., comprises up to 0.5% ABV).

In yet another embodiment, the non-alcoholic beverage of the fifth aspect is an alcohol-free beverage (i.e., comprises up to 0.05% ABV).

Preferably, the non-alcoholic beverage of the fifth aspect, has a pH between 3.0 and 5.0, between 3.0 and 4.5, between 3.0 and 4.0, more preferably a pH above 3.0 and less than 4.5.

In one embodiment, the non-alcoholic beverage of the fifth aspect is a white non-alcoholic beverage, red non-alcoholic beverage, or rose non-alcoholic beverage.

In one embodiment, the non-alcoholic beverage of the fifth aspect is a carbonated or sparkling non-alcoholic beverage, or a still or non-carbonated non-alcoholic beverage.

All of the features described herein (including any accompanying claims, abstracts and drawings), and/or all of the steps of any method or process so disclosed, may be combined with any of the above aspects in any combination, except combinations where at least some features and/or steps are mutually exclusive.

For a better understanding of the invention, and to show how embodiments of the same may be carried into effect, reference will now be made, by way of example, to the accompanying Figures, in which:—

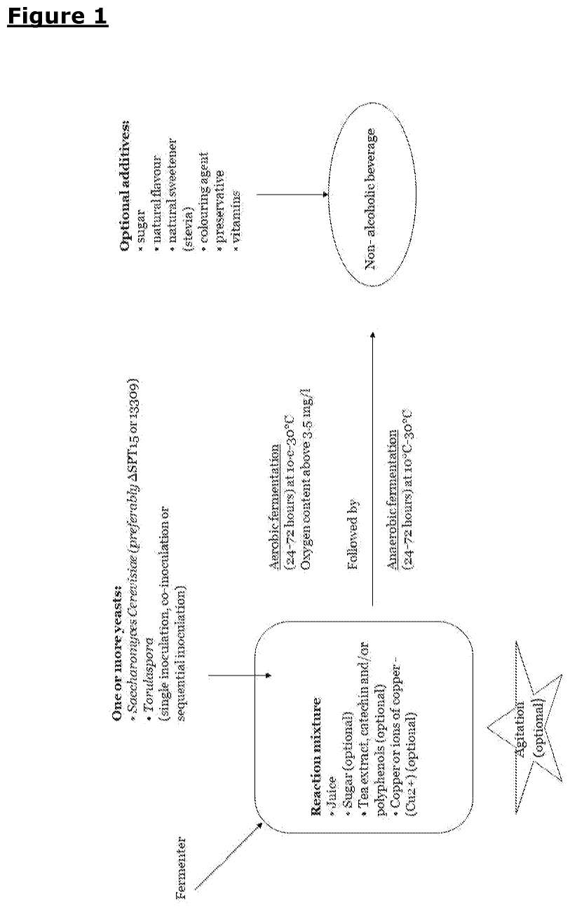

FIG. 1 shows a schematic overview of one embodiment of a method of producing a non-alcohol (i.e., ethanol-reduced or ethanol-free) beverage in which fruit juices or a concentrate thereof are used as the starting material.

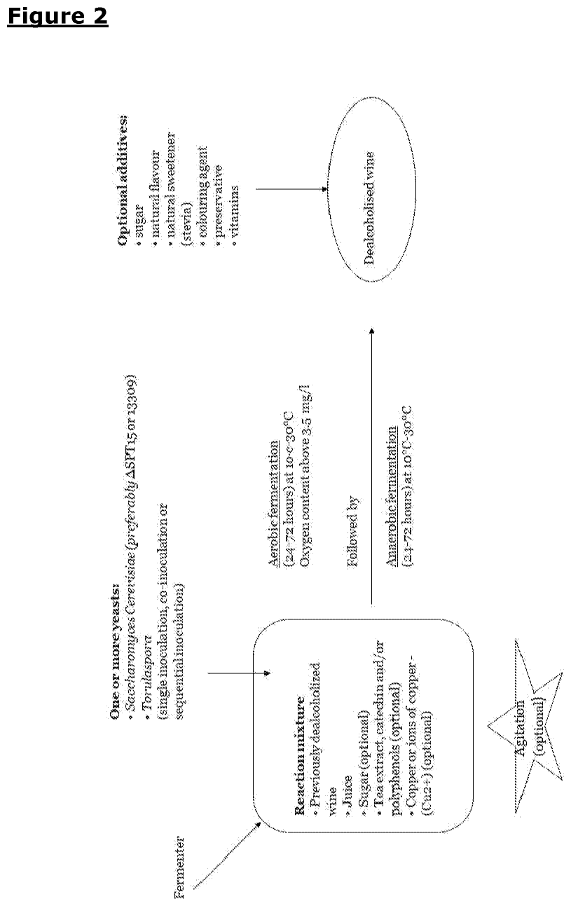

FIG. 2 shows a schematic overview of a first embodiment of a method of producing a dealcoholised (i.e., ethanol-reduced or ethanol-free) wine in which previously dealcoholised wine is used as the starting material.

### EXAMPLES

In response to the increasing need for healthy ethanol-reduced or ethanol-free beverages, such as wines, that provide the health benefits of traditional wine without the negative effects of alcohol, the inventor has made the surprisingly discovery that it is possible to referment previously fermented dealcoholised wine, or to ferment juices or solutions of juices, using a combination of different yeasts, through alternate cycles of aerobic and anaerobic fermentation. Therefore, the inventors have developed and optimised new methods for producing dealcoholised (i.e. ethanol-reduced or ethanol-free) wine and other non-alcoholic beverages (such as juices) with no or very low levels of alcohol in fewer than ten days, while maintaining the standard characteristics, body, flavour, aroma and mouthfeel of traditional alcoholic wine in less than ten days.

### Materials and Methods

### Materials Used

Grape juice concentrate 60-65 Brix (abbreviated GJC)
Apple juice concentrate 65-70 Brix (abbreviated AJC)
Pear juice concentrate 65-70 Brix (abbreviated PJC)
Blackcurrant juice concentrate 65 Brix (abbreviated BCJC)
Blueberry juice concentrate (abbreviated BBJC)
Dealcoholised white wine 0.0% ABV (abbreviated DAWW)
Dealcoholised red wine 0.0% ABV (abbreviated DARW)
Must Microfiltered (abbreviated MM)
Must or grape juice pasteurized (abbreviated MP)
Granular sugar, sucrose (abbreviated GS)
Green tea extracts (abbreviated GTEx)
Wild-type Saccharomyces cerevisiae strain HF 13309
Saccharomyces cerevisiae strain HF 13011
Genetically engineered Saccharomyces cerevisiae of the invention (DSM34577)·
Torulaspora strain HF 13015

### Standard Definitions

Day is abbreviated D. D 1 is day 1, i.e., after 24 hours, D 2 is day 2, i.e., after 48 hours, D 3 is day 3, i.e., after 72 hours, etc.

Percentage of alcohol by volume (ABV).

Volatile sulphur compounds (VSC).

Sensory assessment: 30 ml of liquid is poured in a glass wine container and assessed for the appearance, aroma and palate.

Colour profiling is a visual assessment of the liquid using a white board background.

Smell and aroma attributes are the following: overall intensity, “fruity”: fruit, red fruit, confectionary, “reduced”, rubber, rotten egg, sewage, pungent.

Palate attributes are: fruity, overall fruity, egg taste, sweet, acid, astringent, bitter, and viscosity.

Mouthfeel is an overall sensation in the mouth linked with the body and richness of the liquid.

### Yeast Hydration Protocol

One part of powdered yeast was dissolved in 100 parts of water for 30 minutes.

### Part 1: Fermentation of Juices

Referring to FIG. 1, there is shown a flow chart of an embodiment of the method of producing a non-alcohol beverage in which fruit juices are used as the starting material. First, a reaction mixture comprising one or more fruit juices and optionally sugar, tea extract, catechin and/or polyphenols, copper or ions of copper (Cu²⁺) is put in a fermenter. Subsequently, one or more yeasts (preferably selected from Saccharomyces Cerevisiae and/or Torulaspora) are added to the mixture in either single inoculation, co-inoculation or sequential inoculation. The resulting mixture is then subjected to a combination of aerobic and anaerobic fermentation. After a period ranging from 2 to 8 days, the resulting non-alcohol beverage is produced. Further additives can be added to the resulting wine.

### Example 1: First Fermentation

### Reaction Mixture

The following ingredients were mixed in a fermenter:
Apple Juice Concentrate (AJC): 3.0%
Blackcurrant juice concentrate (BCJC): 2.0%
Granular sugar and sucrose (GS): 1.5%
Green tea extracts (GTEx): 1 g/L
HF13011: 0.2 g/L

The yeast was hydrated for 30 minutes, and the temperature of fermentation was 25° C.

### Results

Day 0: Br 4.8, pH 4.1
D 1: Br 4.3, pH 4.3, ABV: 0.7%
D 2: Br 3.2, pH 3.8, ABV: 1.4%
D 3: Br 2.8, pH 3.7, ABV 1.9%
D 4: Br 2.4, pH 3.7, ABV: 2.3%

### Observations

Overall, the aroma is pleasant with hints of berries and wine notes. The taste and smell of tea are quite apparent.

### Conclusions

Using normal conditions for the fermentation in the presence of Saccharomyces cerevisiae, the inventor observed a conversion of the sugar into alcohol (brix into alcohol %) with a ratio 1 to 1. Every consumption of 1 Brix of sugar generated 1% of alcohol.

### Example 2: Second Fermentation

### Reaction Mixture

The following ingredients were mixed in a fermenter:
Apple juice concentrate (AJC): 2.0%
Pear juice concentrate (PJC): 2.0%
Grape juice concentrate (GJC): 1.0%
Granular sugar, sucrose (GS): 1.5%
Green tea extracts (GTEx): 0.5 g/L
HF 13015 0.4 g/L
HF 13011 0.1 g/L

The yeasts were hydrated for 30 minutes, and the temperature of fermentation was 25° C. The yeasts were inoculated sequentially, with HF13015 first inoculated, followed by the inoculation of HF 13011 24 hours later. Aerobic fermentation was conducted for 3 days.

### Results

D 0: Br 5, pH 4.2
D 1: Br 3.8, pH 3.9, ABV 0.9%
D 2: Br 3.0, pH 3.7, ABV 1.5%
D 3: Br 2.3, pH 3.6, ABV 1.8%

### Observations

Apple and pear aromas are prominent, with hints of wine and bread notes. Low acidity. Even though there is an improvement over the previous non-aerated preparation, the resulting wine still lacks the required body and mouthfeel despite the overall pleasant aroma.

### Conclusions

The sequential inoculation of two different types of yeast with an initial phase of aerobic fermentation using Torulaspora slightly reduced the alcohol content. However, this result was still far from the desired outcome. The agitation during fermentation was not strong enough to generate the desired level of oxygen, given that the oxygen content in the media was around 2 to 3 mg/L.

### Example 3: Third Fermentation

### Reaction Mixture

The Following Ingredients were Mixed in a Fermenter:
Apple juice concentrate (AJC): 2.4%
Blackcurrant juice concentrate (BCJC): 2.0%
Blueberry juice concentrate (BBJC): 1.5%
Grape juice concentrate (GJC): 0.6%
Granular sugar, sucrose (GS): 1.5%
Green tea extracts (GTEx) 0.5 g/L
HF 13015 0.4 g/L
HF 13011 0.1 g/L

The yeasts were hydrated for 30 minutes, and the temperature of fermentation was 25° C. The yeasts were inoculated sequentially, with aerobic fermentation for 24 hours (with HF 13015) followed by anaerobic fermentation (with HF 130110). At the end of day 2, the product was chilled.

### Results

D 0: Br 5, pH 4.2
D 1: Br 3.8, pH 3.7, ABV 0.6%
D 2: Br 3.2, pH 3.6, ABV 0.9%
D 3: Br 2.8, pH 3.6, ABV 1.5%

### Observations

Prominent berry and strawberry aromas with a hint of bread notes. The aroma is pleasant, but the characteristic wine mouthfeel is still lacking.

### Conclusions

Sequential inoculation with first the aerobic fermentation with Torulaspora and then the anaerobic fermentation reduced the alcohol content further. During fermentation, the oxygen content was roughly 2 to 3 mg/L. Reducing the temperature after two days slowed the fermentation process, resulting in an overall decrease in alcohol concentration. Additional reduction of the alcohol content is necessary.

### Example 4: Fourth Fermentation

### Reaction Mixture

The following ingredients were mixed in a fermenter:
Pear juice concentrate (PJC): 2.0%
Apple juice concentrate (AJC): 2.0%
Grape juice concentrate (GJC): 1.0%
Granular sugar, sucrose (GS): 1.5%
Green tea extracts (GTEx) 0.5 g/L
HF 13015 0.4 g/L
HF 13011 0.05 g/L

The yeasts were hydrated for 30 minutes, and the temperature of fermentation was 25° C.

The yeasts were co-inoculated with a lower content of HF 13011. Aerobic fermentation was performed for 24 hours, followed by anaerobic fermentation. From day 3 the product was cooled down.

### Results

D 0: Br 5, pH 4.1
D 1: Br 3.7, pH 3.7, ABV 0.8%
D 2: Br 2.7, pH 3.6, ABV 1.2%
D 3: Br 2.0, pH 3.6, ABV 1.4%

### Observations

The beverage has distinctives notes of pear, apple, and bread, as well as wine. Lack of wine characteristic mouthfeel, despite a pleasing aroma.

### Conclusions

There is no significant difference between co-inoculation and sequential inoculation in terms of flavour profile or alcohol production.

### Example 5: Fifth Fermentation

### Reaction Mixture

The following ingredients were mixed in a fermenter:
Grape juice concentrate (GJC): 3.0%
Blackcurrant juice concentrate (BCJC): 1.0%
Blueberry juice concentrate (BBJC): 1.0%
Apple juice concentrate (AJC): 1.0%
Granular sugar, sucrose (GS): 1.5%
Green tea extracts (GTEx) 0.5 g/L
HF 13015 0.4 g/L

The yeast was hydrated for 30 minutes, and the temperature of fermentation was 25° C.

A single inoculum of Torulaspora was included in the mixture and oxygen was increased through strong agitation during the aerobic phase.

### Results

D 0: Br 4.8, pH 4.2
D 1: Br 3.8, pH 3.6, ABV 0.7%
D 2: Br 3.0, pH 3.5, ABV 1.0%
D 3: Br 2.8, pH 3.5, ABV 1.1%

### Observations

There are fewer wine notes and more juice and a bland aroma. The fermentation was unable to reach completion.

### Conclusions

Torulaspora alone was not sufficient to complete the fermentation with the proper flavour profile, as wine notes were absent. The agitation has been increased, and the oxygen content in the reaction mixture was between 4 and 5 mg/L. A slow start in sucrose consumption was observed during the formation of biomass.

### Example 6: Sixth Fermentation

### Reaction Mixture

The following ingredients were mixed in a fermenter:
Grape juice concentrate (GJC): 1.0%
Blackcurrant juice concentrate (BCJC): 2.0%
Apple juice concentrate (AJC): 1.0%
Green tea extracts (GTEx) 1 g/L
HF 13011 0.4 g/L

The yeast was hydrated for 30 minutes, and the temperature of fermentation was 25° C.

A single inoculum of S. cerevisiae was included in the mixture and anaerobic fermentation was performed.

### Results

D 0: Br 2.8, pH 4.5
D 1: Br 1.8, PH 3.8, ABV 1.0%
D 2: Br 1.6, pH 3.8, ABV 1.2%

### Observations

Although the aroma was pleasant, the mouthfeel was disappointing due to the lower juice content.

### Conclusions

The flavour was fine, but the mouthfeel was disappointing.

### Example 7: Seventh Fermentation

### Reaction Mixture

The following ingredients were mixed in a fermenter:
Grape juice concentrate (GJC): 2.0%
Apple juice concentrate (AJC): 2.0%
Pear juice concentrate (PJC): 2.0%
Granular sugar, sucrose (GS): 1.0%
Green tea extracts (GTEx) 1 g/L
HF 13015 0.4 g/L
HF 13011 0.05 g/L

The yeast was hydrated for 30 minutes, and the temperature of fermentation was 25° C.

Agitation on first 36 hours followed by anaerobic fermentation.

### Results

D 0: Br 4.8, pH 4.2
D 1: Br 4.2, pH 3.9, ABV 0.4%
D 2: Br 3.6, pH 3.8, ABV 0.7%
D 3: Br 2.6, pH 3.7, ABV 1.1%
D 4: Br 2.2, pH 3.7, ABV 1.2%

### Observations

While the flavour profile is pleasant, the mouthfeel should be enhanced. To maintain an ABV of 1.2% or less, the initial aerobic phase must be conducted for 36 hours with an oxygen concentration of at least 4 mg/L.

### Conclusions

During fermentation, the oxygen concentration must remain above 3 mg/L, and vigorous agitation is required to utilise the sugar preventing the production of alcohol. Although the aroma profile was pleasant, the mouthfeel must be enhanced.

### Example 8: Eighth Fermentation

### Reaction Mixture

The following ingredients were mixed in a fermenter:
Grape juice concentrate (GJC): 2.0%
Blackcurrant juice concentrate (BCJC): 2.0%
Apple juice concentrate (AJC): 2.0%
Granular sugar, sucrose (GS): 1.0%
Green tea extracts (GTEx) 1 g/L
HF 13015 0.4 g/L
HF 13011 0.05 g/L

The yeast was hydrated for 30 minutes, and the temperature of fermentation was 25° C.

Agitation on first 36 hours followed by anaerobic fermentation.

### Results

D 0: Br 5.0, pH 4.2
D 1: Br 4.6, pH 3.8, ABV 0.3%
D 2: Br 3.8, pH 3.6, ABV 0.7%
D 3: Br 2.8, pH 3.6, ABV 1.1%
D 4: Br 2.4, pH 3.6, ABV 1.2%

### Observations

While the flavour profile was extremely pleasing, the mouthfeel could be improved. Agitation was carried for 36 hours maintaining the oxygen content above 3 mg/L.

### Conclusions

Strong agitation using both Torulaspora and S. cerevisiae during the initial phase maintained an alcohol concentration of 1.2%. Mouthfeel still needed to be improved and alcohol content should be reduced further.

### Example 9: Ninth Fermentation

### Reaction Mixture

The following ingredients were mixed in a fermenter:
Grape juice concentrate (GJC): 2.5%
Apple juice concentrate (AJC): 1.5%
Pear juice concentrate (PJC): 1.0%
Granular sugar, sucrose (GS): 0.5%
Green tea extracts (GTEx) 0.75 g/L
HF 13015 0.4 g/L
HF 13309 0.1 ml/L

Wild-type S. cerevisiae HF 13309 does not need hydration, whereas the Toluspora HF 13015 was hydrated for 30 minutes, and the temperature of fermentation was 25° C. Fermentation was carried out under aerobic condition (above 4 mg/L of DO) for 36 hours.

### Results

D 0: Br 3.8, pH 3.95
D 1: Br 3.4, pH 3.6, ABV 0.3%
D 2: Br 2.8, pH 3.5, ABV 0.5%
D 3: Br 2.2, pH 3.5, ABV 0.8%

### Observations

There was significant reduction of the content of alcohol but apple aroma and flavours predominated. The mouthfeel remained lacking and needed to be improved.

### Conclusions

The use of wild type S. cerevisiae which produces low levels of alcohol in combination with Torulaspora under agitation in aerated condition reduced the ABV. However, the side effect was a prominent apple taste. Using diluted juices did not improve the mouthfeel.

### Example 10: Tenth Fermentation

### Reaction Mixture

The following ingredients were mixed in a fermenter:
Grape juice concentrate (GJC): 3.0%
Blackcurrant juice concentrate (BCJC): 2.0%
Apple juice concentrate (AJC): 1.0%
Granular sugar, sucrose (GS): 0.5%
Green tea extracts (GTEx): 0.75 g/L
HF 13015:0.4 g/L
HF 13309:0.1 ml/L

Wild-type S. cerevisiae HF 13309 does not need hydration, whereas the mutant S. cerevisiae HF 13015 was hydrated for 30 minutes, and the temperature of fermentation was 25° C. Fermentation was carried out under aerobic conditions (above 3 mg/L of DO) for 36 hours.

### Results

D 0: Br 4.6, pH 3.9
D 1: Br 4.0, pH 3.7, ABV 0.3%
D 2: Br 3.4, pH 3.5, ABV 0.7%
D 3: Br 2.4 pH 3.4, ABV 1.0%

### Observations

There was a significant reduction of the content of alcohol but apple aromas and flavour predominated. Despite the increase in juice content, the mouthfeel was still inadequate and needed to be improved.

### Conclusions

The use of a wild type of S. cerevisiae which produces low level of alcohol in combination with Torulaspora under agitation in aerated condition reduced the amount of ABV further. The side effect was a prominent apple taste. Although there was an increase of juice content with reduction of ABV, there was no significant improvement of the mouthfeel.

### Example 11: Eleventh Fermentation

### Reaction Mixture

The following ingredients were mixed in a fermenter:
Must Microfiltered (MM): 30%
Water (H2O): 70%
HF 13015:0.4 g/L
HF 13011:0.1 g/L

Temperature of fermentation at 23° C., aeration for 24 hours, and then closed the fermenter.

### Results

D 0: Br 7.4, pH 3.9, ABV 0.27%
D 1; 7.0 pH 3.7 ABV 0.5%
D 2: Br 6.2, pH 3.6 ABV 0.9%
D 3: Br 5.2, pH 3.6 ABV 1.2%
D 4: Br 4.4, pH 3.6, ABV 1.6%

### Observations

MM contains 0.8% alcohol by volume (ABV). The must developed a distinct odour of thiols and mercaptans, most likely as a result of the presence of SO2 or a volatile sulphur compound that reacted with Torulaspora. There was a very pleasant Prosecco aroma, but the mouthfeel was still lacking.

### Conclusions

Due to fermentation, the ABV of microfiltered must is 0.8%, and it also contains some yeast, although in small quantities. The odour of thiols or mercaptans generated during fermentation was a result of Torulaspora reacting with SO2 and/or VSC.

The aroma was greatly improved, and was very similar to that of Prosecco, however, the mouthfeel was still weak and watery.

### Example 12: Twelfth Fermentation

### Reaction Mixture

The following ingredients were mixed in a fermenter:
Grape juice concentrate (GJC): 4%
Apple juice concentrate (AJC): 0.5%
Pear juice concentrate (PJC): 0.5%
Green tea extracts (GTEx) 0.5 g/L
Granular sugar, sucrose (GS): 0.5%
DSM34577 dosed 1 ml/L

Temperature of fermentation at 22-25° C., aeration for 24 hours then anaerobic fermentation.

### Results

D 0: Br 3.8, pH 4.6
D 1: Br 3.6, pH 4.4 ABV 0.4%
D 2: Br 3.4, pH 4.3, ABV 0.6%
D 3: Br 3.0, pH 4.2, ABV 0.85%
D 4: Br 2.4, pH 4.1, ABV 1.0%

### Observations

There was a very pleasant scent of fruit and flowers. The fermentation was significantly slower than with HF 13011. Perhaps the biomass had fewer colonies or was somewhat less active. The mouthfeel was light but pleasant.

### Conclusions

The use of DSM34577 yielded comparable result of HF13309 in terms of alcohol formation, but the flavour profile was aromatic with floral and fruity notes. Combining the DSM34577 with Torulaspora could further enhance the wine in terms of flavour and alcohol reduction.

### Example 13: Thirteenth Fermentation

### Reaction Mixture

The following ingredients were mixed in a fermenter:
Grape juice concentrate (GJC): 3%
Green tea extracts (GTEx): 0.5 g/L
Granular sugar, sucrose (GS): 0.3%
HF 13015:0.3 g/L
DSM34577 dose 2 ml/L

Temperature of fermentation at 22-25° C., aeration for 36 hours, and then anaerobic fermentation.

### Results

D 0: Br 2.4, pH 4.6
D 1: Br 2.4, pH 4.4 ABV 0.1%
D 2: Br 2.2, pH 4.3 ABV 0.25%
D 3: Br 2.0, pH 4.2 ABV 0.4%

### Observations

The aroma and flavour profile was pleasant with floral and fruity notes. The mouthfeel was light.

### Conclusions

The alcohol content had been reduced to less than 0.5%, which was the overall goal and a pleasant wine with a delightful aroma of fermented floral fruity notes was created. Further optimisation of the genetically engineered yeast will then increase the content of juice. However, the mouthfeel required further improvements.

### Example 14: Fourteenth Fermentation

### Reaction Mixture

The following ingredients were mixed in a fermenter:
Grape juice concentrate (GJC): 3%
Apple juice concentrate (AJC): 0.5%
Green tea extracts (GTEx): 0.75 g/L
Granular sugar, sucrose (GS): 0.3%
HF 13015:0.4 g/L
DSM34577 dose 1 ml/L

Temperature of fermentation at 22-25° C., aeration for 48 hours, and then anaerobic fermentation.

### Results

D 0: Br 2.5, pH 4.5
D 1: Br 2.4, pH 4.4 ABV 0.1%
D 2: Br 2.2, pH 4.3 ABV 0.2%
D 3: Br 2.0, pH 4.2 ABV 0.4%

### Observations

The aroma and flavour profile was pleasant with floral and fruity notes. The mouthfeel was light.

### Conclusions

The alcohol content had been reduced to less than 0.5%, which was the overall goal and a pleasant beverage with a delightful aroma of fermented floral fruity notes was created. Further optimisation of the genetically engineered yeast will then increase the content of juice. However, the mouthfeel required further improvements.

### Part 2: Fermentation of Dealcoholised Wine

To compensate for the lack of body and mouthfeel that was observed with the fermentation of solution of juices in Examples 1-14, the inventor considered using previously dealcoholised wines as the starting material. Standard dealcoholised wines are completely devoid of flavour because the volatile and low molecular weight components have been eliminated during the dealcoholisation process, while heavy molecular weight and water-soluble components remain, resulting in a wine that lacks flavour and has an altered taste.

The inventor has now found that these wines retain some of the deleterious sulphur compounds that provide the typical “reduced” aroma and flavour of rubber, sewage, stewed vegetables, or other pungent compounds, as well as the polyphenols that are typically bitter in taste and various acids that are extremely astringent. Through refermentation, the inventors therefore hoped to eradicate at least a portion of these negative sulphur compounds that are present in dealcoholized wines and impart a floral, fruity aroma derived from the yeast compounds produced during fermentation. Compounds, such as acetate- and ethyl-esters, contribute to the fruity aroma of the dealcoholised wine of the present invention.

Surprisingly, the inventors discovered that the sulphur compounds can be broken down and removed from the media using copper ions (Cu2+) as a treatment to eliminate “reductive” aromas during fermentation, thanks to a refermentation step conducted in the presence of oxygen and with various types of yeast.

Referring to FIG. 2, there is shown a flow chart of another embodiment of the method of producing a dealcoholised wine in which previously dealcoholised wine is used as the starting material. First, a reaction mixture comprising the previously dealcoholised wine and one or more fruit juices, and optionally sugar, tea extract, catechin and/or polyphenols, copper or ions of copper (Cu²⁺) is put in a fermenter. Subsequently, one or more yeasts (preferably selected from Saccharomyces Cerevisiae and/or Torulaspora) are added to the mixture in either single inoculation, co-inoculation or sequential inoculation. The resulting mixture is then subjected to a combination of aerobic and anaerobic fermentation. After a period ranging from 2 to 8 days, the resulting dealcoholised wine is produced. Further additives can be added to the resulting wine.

### Example 15: Fifteenth Fermentation

### Reaction Mixture

The following ingredients were mixed in a fermenter:
Dealcoholised white wine 0.0% ABV (DAWW): 80%
Must Microfiltered (MM): 18%
Grape juice concentrate (GJC): 2%
HF 13011:0.2 g/L

Temperature of fermentation is 25° C., agitation for 24-36 hours. DAWW Br 2.2 and pH 3.5. MM has initial ABV of 0.8% and GJC has been added on day 2.

### Results

D 0: Br 5.2, pH 3.6 ABV 0.15%
D 1: Br 4.8, pH 3.5, ABV 0.2%
D 2: Br 4.5, pH 3.45, ABV 0.5% (with an addition of juice)
D 3: Br 4.7, pH 3.5 ABV 1.1%
D 4: Br 3.0, pH 3.5 ABV 1.2%

### Observations

There was the development of a thiols/mercaptans aroma that affected the overall perception of the wine. The mouthfeel has significantly improved and it closely resembled that of standard wine and Prosecco. The aroma of thiols prevented an accurate evaluation of flavour.

### Conclusions

The increase in ABV is consistent with the fermentation of juices, and the mouthfeel of the resulting product closely resembled that of wine. As a result of the VSC (volatile sulphur compound) that has been produced, the odour evolution cannot be completely evaluated. It appears that Torulaspora promptly converts the VSCs present in the de-alcoholized wine, as the amount of VSCs is lower than in previous tests.

### Example 16: Sixteenth Fermentation

### Reaction Mixture

The following ingredients were mixed in a fermenter:
Dealcoholised white wine 0.0% ABV (DAWW): 95%
Grape juice concentrate (GJC): 3.0%
Apple juice concentrate (AJC): 1.0%
Pear juice concentrate (PJC): 0.5%
GS: 0.5%
HF 13011:0.2 g/L

Temperature of fermentation is between 22-25° C. The reaction is aerated for 24 hours. DAWW Br 2.2 and pH 3.5.

### Results

D 0: Br 5.2, pH 3.6, ABV 0.0%
D 1: Br 4.8, pH 3.5 ABV 0.3%
D 2: Br 4.2, pH 3.5 ABV 0.5%
D 3: Br 3.8 pH 3.5 ABV 0.7%
D 4: Br 3.5, pH 3.5 ABV 0.85%

### Observations

The flavour profile was quite pleasant as a result of the refermentation. There were fruit and apple notes present.

### Conclusions

Compared to the fermentation of a solution of juice, the mouthfeel can be surprisingly enhanced by beginning with dealcoholised wine. The aroma was enhanced, but there was still a trace of VSC in the resulting wine, as well as bitterness and astringency that needed to be compensated for. A post-addition of sugar can mitigate these negative effects.

### Example 17: Seventeenth Fermentation

### Reaction Mixture

The following ingredients were mixed in a fermenter:
Dealcoholised white wine 0.0% ABV (DAWW): 90%
Grape juice concentrate (GJC): 4.0%
Apple juice concentrate (AJC): 0.5%
Water (H₂O): to 100%
Granular sugar, sucrose (GS): 0.3%
HF 13015:0.3 g/L
DSM34577: 10.4 dose 1 ml/L
Cu²⁺0.4 mg/L

Temperature of fermentation is between 22-25° C. The reaction was aerated for 36 hours. DAWW: Br 2.2 and pH 3.9.

### Results

D 0: Br 5.2 pH 3.9
D 1: Br 5.0 pH 3.8 ABV 0.1%
D 2: Br 4.8 pH 3.8 ABV 0.2%
D 3: Br 4.4 pH 3.8 ABV 0.25%
D 4: Br 3.8 pH 3.8 ABV 0.5%
D 5: Br 3.4 pH 3.8 ABV 0.7%

### Observations

The flavour profile was pleasant with floral and fruity notes. There was no reduction in smell using the aeration and preventive treatment with Cu²⁺. The optimal wine taste was reached.

### Conclusions

The mouthfeel was very good and mirrored normal wine, and the aroma was excellent. Due to the refermentation, the aroma and flavour of VSC, which are characteristic of dealcoholised wine, were substantially diminished, and the resulting flavour and aroma were characteristic of wine. A tiny amount of rectified concentrated juice will counterbalance the residual acidity and polyphenolic effect. By slightly reducing the quantity of starting sugar, the final alcohol content can be reduced below 0.5%.

### Example 18: Eighteenth Fermentation

### Reaction Mixture

The following ingredients were mixed in a fermenter:
Dealcoholised white wine 0.0% ABV (DAWW): 88%
Must or grape juice pasteurized (MP): 10%
Grape juice concentrate (GJC): 1.5%
Granular sugar, sucrose (GS): 0.3%
HF13015: 0.3 g/L
DSM34577: dose 1 ml/L
Cu²⁺ 0.4 mg/L

Temperature of fermentation is between 22-25° C. The reaction was aerated for 36 hours to maintain a dissolved oxygen of minimum 3 mg/L, preferably above 4 mg/L. DAWW: Br 2.2 and pH 3.8.

### Results

D 0: Br 4.9 pH 3.8
D 1: Br 4.7, pH 3.8 ABV 0.1%
D 2: Br 4.5, pH 3.7 ABV 0.2%
D 3: Br 4.0, pH 3.7 ABV 0.4%
D 4: Br 3.4, pH 3.7 ABV 0.6%

### Observations

Very pleasant flavour with fruity floral aroma. Mouthfeel resembled that of traditional wine.

### Conclusions

The overall flavour, aroma, taste, mouthfeel and aftertaste were remarkably similar to traditional wine and superior to any dealcoholised product available in the market that has been sampled.

### Example 19: Ninetieth Fermentation

### Reaction Mixture

The following ingredients were mixed in a fermenter:
Dealcoholised white wine 0.0% ABV (DAWW): 90%
Must or grape juice pasteurized (MP): 6.25%
Grape juice concentrate (GJC): 2%
DSM34577: dose 2 ml/L
Cu2+ 0.4 mg/L

Temperature of fermentation is between 22-25° C. The reaction was aerated for 24 hours. DAWW: Br 2.2 and pH 3.8.

### Results

D 0: Br 4.5 pH 3.8
D 1: Br 4.4, pH 3.8 ABV 0.0%
D 2: Br 4.0, pH 3.7 ABV 0.2%
D 3: Br 3.2-, pH 3.7 ABV 0.45%

### Observations

Very pleasant flavour with fruity floral aroma. Mouthfeel resembled that of traditional wine.

### Conclusions

The overall flavour, aroma, taste, mouthfeel and aftertaste were remarkably similar to traditional wine and superior to any de-alcoholized product available in the market that has been sampled.

### Example 20: Twentieth Fermentation

### Reaction Mixture

The following ingredients were mixed in a fermenter:
Dealcoholised white wine 0.0% ABV (DAWW): 90%
Must or grape juice pasteurized (MP): 6.25%
Grape juice concentrate (GJC): 2%
DSM34577: dose 1 ml/L
Cu2+ 0.4 mg/L

Temperature of fermentation is between 22-25° C. The reaction was aerated for 48 hours. DAWW: Br 2.2 and pH 3.8.

### Results

D 0: Br 4.5 pH 3.8
D 1: Br 4.4, pH 3.8 ABV 0.0%
D 2: Br 4.0, pH 3.7 ABV 0.15%
D 3: Br 3.2-, pH 3.7 ABV 0.4%

### Observations

Very pleasant flavour with fruity floral aroma. Mouthfeel resembled that of traditional wine.

### Conclusions

The overall flavour, aroma, taste, mouthfeel and aftertaste were remarkably similar to traditional wine and superior to any dealcoholized product available in the market that has been sampled.

### SUMMARY

The inventor has developed and optimised new methods for producing de-alcoholised wine with no or very low levels of alcohol (i.e., less than 0.5% ABV) while maintaining the standard characteristics, body, flavour, aroma and mouthfeel of traditional alcoholic wine, or other non-alcoholic beverages with no or very low levels of alcohol (i.e., less than 1.2% ABV). He has achieved this by either refermenting previously fermented wine, optionally with added juices or concentrated juices, or by fermenting juices or solutions of juices or concentrated juices, using a combination of different yeast species. In particular, the inventor used genetically engineered Saccharomyces cerevisiae yeasts, through alternate cycles of aerobic and anaerobic fermentation.

1. A method for producing a de-alcoholised wine, the method comprising re-fermenting a reaction mixture comprising a previously de-alcoholised wine with one or more yeasts, thereby producing the de-alcoholised wine.

2. The method of claim 1, wherein the previously de-alcoholised wine represents between 70% and 98% (v/v) of the reaction mixture, or between 80% and 98%, or between 85% and 95% (v/v) of the reaction mixture.

3. The method of claim 1 or 2, wherein the reaction mixture further comprises one or more fruit juices or one or more fruit juice concentrates, optionally wherein the fruit is selected from grape, grape must, apple, pear, blackcurrant, berry, and/or mango, and optionally wherein the one or more fruit juice is provided in a concentration of less than 20% (v/v) of the reaction mixture.

4. The method of any preceding claim, wherein the reaction mixture further comprises granular sugar, simple sugar, monosaccharides, and/or disaccharides.

5. The method of any preceding claim, wherein the total sugar content of the reaction mixture is between 2 and 15° Br, optionally between 3 and 10° Br.

6. The method of any preceding claim, wherein the reaction mixture further comprises tea extract, catechin, polyphenols, and/or copper or ions of copper (Cu²⁺),

7. The method of claim 6, wherein the tea extract, catechin and/or polyphenols are provided at a concentration of between 0.25 g/L and 2 g/L, and/or the copper or ions of copper are provided at a concentration of between 0.25 g/L and 2 g/L.

8. The method of any preceding claim, wherein the one or more yeasts is selected from a group of yeast genuses consisting of: Saccharomyces, Torulaspora, Lachancea, Metschnikowia, and Schizosaccharomyces, or a combination thereof; preferably a yeast of the genus, Saccharomyces.

9. The method of any preceding claim, wherein the one or more yeasts comprises a Saccharomyces cerevisiae.

10. The method of any preceding claim, wherein the previously de-alcoholised wine is re-fermented with a genetically engineered yeast strain comprising a mutated SPT-15 gene, or a homologue or paralogue thereof, a wild-type of S. cerevisiae HF 13011, and/or wild-type of S. cerevisiae HF 13309.

11. The method of claim 10, wherein the mutated SPT-15 gene encodes an amino acid sequence comprising mutations at at least three positions selected from: I46, D56, S118, Y195, and L205 in the amino acid sequence of the wild-type S. cerevisiae SPT-15 gene, optionally wherein the mutations at positions I46, D56, S118, Y195, and L205 are I46M, D56G, S118P, Y195H, and L205S, respectively,
optionally wherein the yeast strain comprising the mutated SPT-15 gene is of the genus selected from the group consisting of: Saccharomyces spp., Kazachstani spp., Torulaspora spp, and Pichia spp., preferably S. cerevisiae, and
optionally wherein the S. cerevisiae expressing a mutated SPT-15 gene comprises at least one genomic copy of the mutated SPT-15 gene, and the at least one copy of the mutant SPT-15 gene is inserted into the genomic ADH-1 gene locus.

12. The method of claims 10 and 11, wherein the S. cerevisiae expressing a mutated SPT-15 is S. cerevisiae strain BR 23007 deposited under DSM 34577 (DSM34577).

13. The method of any preceding claim, wherein the one or more yeasts further comprises a yeast of the genus, Toluraspora, optionally wherein the Torulaspora is T. delbrueckii, and optionally T. delbrueckii wildtype wine yeast HF 13015.

14. The method of any preceding claim, wherein the reaction mixture is inoculated with a single yeast strain or species or with at least two yeast strains or species, optionally wherein the at least two yeast strains comprise Saccharomyces spp, and Torulaspora spp., preferably, S. cerevisiae and T. delbrueckii, more preferably, the S. cerevisiae is selected from the wild-type S. cerevisiae strain HF 13309, the wild-type S. cerevisiae strain HF 13011 or S. cerevisiae strain DSM34577, and the Torulaspora spp. is Torulaspora strain HF 13015.

15. The method of claim 14, wherein the one or more yeasts are co-inoculated, or sequentially inoculated.

16. The method of any preceding claim, wherein the total amount of yeast inoculated in the reaction mixture is at a concentration of between 0.1 g/L and 5 g/L, optionally between 0.1 g/L and 3 g/L, or between 10⁸ and 10⁹ cfu.

17. The method of any preceding claim, wherein the re-fermentation of the reaction mixture takes place at a temperature of between 10° C. and 30° C., or between 20° C. and 25° C.

18. The method of any preceding claim, wherein the re-fermentation of the reaction mixture is carried out in fewer than 15 days, preferably between 2 and 10 days.

19. The method of any preceding claim, wherein the re-fermentation of the reaction mixture comprises a first phase of aerobic fermentation and a second phase of anaerobic fermentation, preferably wherein the first phase of aerobic fermentation is followed by a second phase of anaerobic fermentation.

20. The method of claim 19, wherein the aerobic fermentation is conducted for 12 to 72 hours, optionally at a temperature of between 10° C. and 30° C., preferably between 20° C. and 25° C.

21. The method of claim 20, wherein dissolved oxygen content is at least 2.5 mg/L, optionally above 3.5 mg/L.

22. The method of any one of claim 19-21, wherein the anaerobic fermentation is conducted for 12 to 72 hours, optionally at a temperature of between 10° C. and 30° C., preferably between 20° C. and 25° C.

23. A de-alcoholised wine obtained, or obtainable, by the method of any one of claim 1-22.

24. The de-alcoholised wine of claim 23, wherein the de-alcoholised wine further comprises sugar, natural flavour, natural sweetener, colouring agent, preservative, and/or vitamins added after the re-fermentation.

25. The de-alcoholised wine of claim 23 or 24, wherein the de-alcoholised wine is a low-alcohol de-alcoholised wine (i.e., comprises up to 1.2% ABV), a non-alcoholic de-alcoholised wine (i.e., comprises up to 0.5% ABV), or an alcohol-free de-alcoholised wine (i.e., comprises up to 0.05% ABV).

26. The de-alcoholised wine of any one of claims 23-25, wherein the de-alcoholised wine has a pH between 3.0 and 5.0, optionally less than 4.5.

27. The de-alcoholised wine of any one of claims 23-26, wherein the de-alcoholised wine is a white wine, red wine, or rose wine, optionally wherein the de-alcoholised wine is a carbonated or sparkling wine, or a still or non-carbonated wine.

28. A method for producing a reduced alcohol or non-alcoholic beverage, the method comprising fermenting a reaction mixture comprising one or more fruit juices, or one or more fruit juice concentrates, with one or more yeasts, wherein the one or more yeasts comprises a genetically engineered yeast strain comprising a modified SPT-15 gene or a homologue or paralogue thereof, or wild-type Saccharomyces cerevisiae strain HF 13309, to thereby produce a reduced alcohol or non-alcoholic beverage.

29. The method of claim 28, wherein the one or more fruit juice, or one or more fruit juice concentrate makes up less than up less than 20% (v/v) of the fermentation reaction mixture, optionally less than 15% (v/v) of the reaction mixture.

30. The method of claim 28 or 29, wherein the fruit is selected from grape, grape must, apple, pear, blackcurrant, berry, and/or mango.

31. The method of any one of claims 28-30, wherein the reaction mixture further comprises the features recited in any one of claims 4-7.

32. The method of any one of claims 28-31, wherein the mutated SPT-15 gene is as described in any one of claims 10-12.

33. The method of any one of claims 28-32, wherein the one or more yeasts further comprises a yeast selected from a group of yeast genuses consisting of: Saccharomyces, Torulaspora, Lachancea, Metschnikowia, and Schizosaccharomyces, or a combination thereof.

34. The method of any one of claims 28-33, wherein the one or more yeasts further comprises a yeast from the genus Saccharomyces, optionally S. cerevisiae HF 13011 and/or a yeast of the genus, Toluraspora, preferably, the Torulaspora is T. delbrueckii, more preferably T. delbrueckii wildtype wine yeast HF 13015.

35. The method of any one of claims 28-34, wherein the reaction mixture is inoculated as described in any one of claims 14-16.

36. The method according to claim 28, wherein the fermentation is carried out under the conditions described in any one of claims 17-22.

37. A non-alcoholic beverage obtained, or obtainable, by the method obtained, or obtainable, by the method of any one of claims 28-36.

38. The non-alcoholic beverage of claim 37, wherein the non-alcoholic beverage exhibits the features and characteristics described in any one of claims 24-27.

39. A non-alcoholic beverage comprising the de-alcoholised wine of any one of claims 23-27 and the non-alcoholic beverage of either claim 37 or 38.

40. The non-alcoholic beverage of claim 39, wherein the non-alcoholic beverage comprises approximately an equal amount (i.e., 50% v/v) of each of the de-alcoholised wine of any one of claims 23-27 and the non-alcoholic beverage of either claim 37 or 38.

41. The non-alcoholic beverage of claim 39 or 40, wherein the non-alcoholic beverage comprises 10% (v/v) of the de-alcoholised wine of any one of claims 23-27 and 90% (v/v) of the non-alcoholic beverage of either claim 37 or 38, preferably 20% (v/v) of the de-alcoholised wine of any one of claims 23-27 and 80% (v/v) of the non-alcoholic beverage of either claim 37 or 38, or 30% (v/v) of the de-alcoholised wine of any one of claims 23-27 and 70% (v/v) of the non-alcoholic beverage of either claim 37 or 38, or 40% (v/v) of the de-alcoholised wine of any one of claims 23-27 and 60% (v/v) of the non-alcoholic beverage of either claim 37 or 38, or 60% (v/v) of the de-alcoholised wine of any one of claims 23-27 and 40% (v/v) of the non-alcoholic beverage of either claim 37 or 38, or 70% (v/v) of the de-alcoholised wine of any one of claims 23-27 and 30% (v/v) of the non-alcoholic beverage of either claim 37 or 38, or 80% (v/v) of the de-alcoholised wine of any one of claims 23-27 and 20% (v/v) of the non-alcoholic beverage of either claim 37 or 38, or 90% (v/v) of the de-alcoholised wine of any one of claims 23-27 and 10% (v/v) of the non-alcoholic beverage of either claim 37 or 38.

42. The non-alcoholic beverage of claim 41, wherein the non-alcoholic beverage comprises 90% (v/v) of the de-alcoholised wine of any one of claims 23-27 and 10% (v/v) of the non-alcoholic beverage of either claim 37 or 38.

43. The non-alcoholic beverage of any one of claims 39-43, wherein the non-alcoholic beverage exhibits the features and characteristics described in any one of claims 24-27.
